(19) United States
(12) Patent Application Publication
(10) Pub. No.: US 2004/0072729 A1
(43) Pub. Date: Apr. 15, 2004

(54) High oxygen affinity PEG-hemoglobin as treatment for brain stroke

(75) Inventors: Nho Kwang, Walnut Creek, CA (US); Park Minkoo, Anyang-si (KR); Lee Ji-Won, Anyang-si (KR)

Correspondence Address: KNOBBE MARTENS OLSON & BEAR LLP, 2040 MAIN STREET, FOURTEENTH FLOOR, IRVINE, CA, 92614, US

(73) Assignee: SunBio Inc.

(21) Appl. No.: 10/267,833

(22) Filed: Oct. 10, 2002

(51) Int. Cl. A61K 38/42

(52) U.S. Cl. 514/6

(57) ABSTRACT

The invention relates to a method for treating symptoms such as stroke comprising administering PEG-hemoglobin (polyethylene glycol-conjugated hemoglobin) solution with specific ability to deliver oxygen to cells under ischemia or hypoxia.

The PEG-hemoglobin of the present invention is an oxygen carrier having demonstrated therapeutic efficacy in treatment of brain strokes. The PEG-hemoglobin disclosed in this invention has a unique ability to unload most of the carried oxygen at the site of very low oxygen milieu. This characteristic is attributed to the relatively low p50 value (8-12 mmHg) of this invention. The invention is readily applicable to restore normal oxygen level for oxygen-deprived cells such as ischemic brain cells under stroke.

### FIELD OF THE INVENTION

The present invention relates to a method for treating symptoms such as stroke comprising administering polyethylene glycol-conjugated hemoglobin solution.

### BACKGROUND OF THE INVENTION

Stroke is the third leading cause of death, behind diseases of the heart and cancer and the main cause of adult disability. It is now estimated that there are more than 700,000 stroke incidents occurring annually and 4.4 million stroke survivors in the U.S.A. (Heart and Stroke Statistical Update. Dallas, Tex.: American Heart Association, 2000). There are two main categories of stroke, i.e., ischemic and hemorrhagic stroke. Ischemic strokes account for 83% of all strokes, and occur as either a thrombotic or embolic stroke. Thrombotic strokes represent 52% of all ischemic strokes. An ischemic stroke can be caused by blood clots forming inside the artery of the brain (thrombotic stroke), or by clots forming elsewhere in the body and subsequently reaching the brain (embolic stroke). Ischemic strokes are commonly caused by atherosclerosis, a buildup of plaque inside the arteries. A hemorrhagic stroke occurs when an artery in the brain leaks or ruptures. The hemorrhaged blood pressures the surrounding tissue, subsequently leading to blockage of blood flow to the brain cells. Brain cells beyond the hemorrhagic site become deprived of oxygen and thus cause to ischemia. The most common cause of a hemorrhagic stroke is high blood pressure. The constant force of high blood pressure can weaken blood vessel walls, resulting in hemorrhage. Another cause of a hemorrhagic stroke is leakage from an aneurysm. An aneurysm is a weak spot in an artery wall that becomes thin and stretched. If the aneurysm ruptures, hemorrhage will ensue. A transient ischemic attack (TIA) is a temporary interruption of blood flow to parts of the brain, also referred to as a mini-stroke. The symptoms for TIA are the same as for a stroke, but they appear for a shorter period of time (several minutes to 24 hours) and then disappear (1995-2002 Mayo Foundation for Medical Education and Research, http://www.mayo.edu). The interruption of cerebral blood flow deprives brain cells of oxygen, leading to a reduction in energy production and an associated build-up of toxic metabolites that trigger the ischemic cascade. This involves the release of mediators of cell death, including the neurotransmitter glutamate, inflammatory mediators and reactive oxygen species (ROS). Ultimately, these secondary reactions result in brain cell damage and death (Martina Habeck, Drug Discovery Today, 7(3) p157, 2002). New treatment approaches target one of these mechanisms including anti-oxidant therapy to inhibit ROS, anti-inflammatory therapy to inhibit cyclo-oxygenase (COX)-2 activity as well as chemokine and cytokine production, and anti-apoptosis therapy to prevent neuronal apoptosis by blocking one of intracellular signaling pathways. But the only medical treatment that is available to date is the administration of tissue plasminogen activator (t-PA) that can dissolve blood clots if administered within three hours after the onset of the stroke. However, this treatment is limited in availability and efficacy as this time restraint renders over 90% of cases in the U.S.A. untreatable. Therefore, there is an urgent need for emergency treatment therapies that can restore and maintain normal tissue oxygen level for brain cells under ischemia.

Hemoglobin, normally a tetramer of two α and two β chains weighing 64.5 kDa, when outside of the red blood cell, dissociates into molecular fragments, αβ1 and α2β2, with a molecular weight of 32 kDa each, and is thus freely filtered by the kidney. As a result, cell-free hemoglobin has a short half-life due in most part to renal excretion and also due to clearance by the reticuloendothelial system.

Oxygen carriers, or blood substitutes fall into two main categories: hemoglobin-based oxygen carriers (HBOCs) and perfluorocarbon-based oxygen carriers. HBOCs utilize modified human, animal, or recombinant hemoglobin as hemoglobin source. In contrast, perfluorocarbon emulsions are concentrated emulsions of chemical particles suspended in a water-based solution. The HBOCs are in most cases modified hemoglobins, and these hemoglobins are in most cases crosslinked or polymerized to endow structural stability and extended intravascular circulation time. HBOCs are known to bind the nitric oxide (NO), endogenous vasodilator. Locally released NO has been known to be scavenged by hemoglobin thus preventing NO from exerting a tonic vasodilator action, thereby allowing vasoconstrictor mechanisms to dominate. This may result in an increase in blood pressure in patients treated with HBOCs. These hemodynamic effects may be transient and may respond adequately to medical treatment (F. J. Lou Carmichael, Transfusion and Apheresis Science, 24, pp 17-21, 2001 and F. J. Lou Carmichael et al., Blood, 94, pp 116b-7b, 1999).

There are several HBOCs that are under development. Hemolink™ is a human hemoglobin-based oxygen carrier which is a crosslinked and polymerized hemoglobin by o-raffinose. PolyHeme™ is a human-derived hemoglobin polymerized with glutaraldehyde. Hemopure® is a bovine hemoglobin polymerized with glutaraldehyde. Apart from the above-mentioned crosslinked or polymerized hemoglobins, some have been developing PEG-hemoglobins.

Polyethylene glycol (PEG), is a water-soluble polymer that exhibits properties such as low toxicity, non-immunogenicity and ready availability in a variety of molecular weights. The structure of PEG is illustrated as the following: HO—(—CH₂CH₂O—)ₙ—H. PEG in solution typically binds 2-3 water molecules (H₂O) per ethylene oxide (—CH₂CH₂O—) unit. Due to both the high flexibility of the backbone chain and the binding of water molecules, the PEG molecule acts as if it were 5-10 times as large as a soluble protein of comparable molecular weight (M. J. Roberts et al., Advanced Drug Delivery Reviews, 54, pp 459-476, 2002). PEG has been found to be nontoxic and is approved by the FDA for use in drugs (parenterals, topicals, suppositories and nasal sprays), foods, and cosmetics (F. Fuertges A. and A. Abuchowski, J. Control. Release, 11, pp 139-148, 1990).

Parenterally administered pharmaceutically useful proteins may be immunogenic or be relatively water insoluble and may have a short in vivo half-life. Modification of the polypeptide with a polymer such as PEG has been shown to be efficacious as a means of overcoming these problems and thus enhancing the protein's therapeutic efficacy (J. Milton Harris, 1992, Poly(Ethylene Glycol)). U.S. Pat. No. 4,179,337 discloses conjugating PEG to proteins such as enzymes and insulin to provide a less immunogenic product while retaining a substantial proportion of the biological activity. The modification of ribonuclease and superoxide dismutase with PEG has been described (Veronese et al., Applied Biochem. and Biotech., 11, pp 141-152, 1985). U.S. Pat. Nos. 4,766,106 and 4,917,888 also disclose examples of solubilized proteins by polymer conjugation. PEG and other polymers are also conjugated to recombinant proteins to reduce immunogenicity and to increase half-life (Nitecki et al., U.S. Pat. No. 4,902,502).

The most preferred molecular weight range of PEG utilized in the present invention is between 1,000 and 100,000. The toxicity is substantially lower when PEG having a molecular weight over 1,000 is used. The PEG having molecular weights ranging from about 1,000 to 6,000 is generally known to be distributed through the whole body, and metabolized by the kidney (C. B. Shaffer et al., J. Am. Pharm. Assoc., 36, p 152, 1947).

Many attempts to overcome the short plasma half-life of pharmaceutically useful proteins have been tried. Some of the successful examples show that enhanced pharmacokinetic properties can be achieved by PEG modification (Francis et al., J. Drug Target, 3, pp 321-340, 1996). The enhanced effects that could be acquired by PEG modification may differ depending on the target protein. And more significantly the effects can vary depending on the site of PEG attachment to the protein, the chemistry used for generating the conjugate, and the physical properties of the PEG itself such as size, structure, etc. (Delgado et al., Pharm. Sci., 3, pp 59-66, 1997).

There have been attempts to develop PEG-hemoglobins. As a result, there appeared several different formulations of PEG-hemoglobin that differ in the type of PEG derivatives used to modify hemoglobin, and also differ in the value of p50. The p50 is defined as the partial pressure of oxygen (mmHg) at which point 50% of the hemoglobin is oxygenated. The p50 of normal blood red blood cells is 26-28 mmHg. Lower p50 such as 8-12 mmHg represents a stronger affinity of hemoglobin for oxygen, thereby the oxygenated hemoglobin is more reluctant to release oxygen.

As summarized in Table 1, U.S. Pat. No. 4,412,989 disclosed a PEG-hemoglobin in which mPEG-succinimidyl succinate (SS-PEG) was used for modification of hemoglobin. The value of p50 was not determined. U.S. Pat. No. 4,670,417 disclosed a PEG-hemoglobin in which mPEG-succinimidyl succinate (SS-PEG) was used for modification of hemoglobin. The value of p50 was widely dispersed to be in the range of 6.1-19.0 mmHg. U.S. Pat. No. 5,234,903 disclosed a PEG-hemoglobin in which mPEG-succinimidyl carbonate (SC-PEG) was used for modification of hemoglobin. The value of p50 was greater than 20 mmHg. U.S. Pat. No. 5,478,806 disclosed a utility of the PEG-hemoglobin as a cancer sensitizer. The PEG-hemoglobin was modified with mPEG-succinimidyl carbonate (SC-PEG), and the value of p50 was greater than 20, as the PEG-hemoglobin referred to be identical with that in U.S. Pat. No. 5,234,903. U.S. Pat. No. 6,054,427 disclosed a PEG-hemoglobin in which mPEG-malemide (MAL-PEG) was used for modification of hemoglobin. The value of p50 was less than 28 mmHg.

TABLE 1
Characteristics of PEG-hemoglobins Disclosed in U.S. Pat. No.

| U.S. Pat. No. | Type of PEG Derivative Used | p50 (mmHg) |
|---|---|---|
| 4,412,989 (11/1983) | mPEG-succinimidyl succinate | NA. |
| Iwashita el al. | (SS-PEG) | |
| 4,670,417 (6/1987) | mPEG-succinimidyl succinate | 6.1-19.0 |
| Iwasaki et al. | (SS-PEG) | |
| 5,234,903 (8/1993) | mPEG-succinimidyl carbonate | Greater than 20 |
| Nho et al. | (SC-PEG) | |
| 5,478,806 (12/1995) | mPEG-succinimidyl carbonate | Greater than 20 |
| Nho | (SC-PEG) | |
| 6,054,427 (4/2000) | mPEG-maleimide | Less than 28 |
| Winslow | (MAL-PEG) | |

There also have been several different types of HBOCs without the use of PEG as a modifier. As summarized in Table 2, U.S. Pat. No. 4,831,012 disclosed a structurally stabilized form of hemoglobin wherein the subunits of hemoglobin were linked together with a small diaspirin molecule to prevent the hemoglobin subunits from breaking apart, and p50 was reported to be in the range of 20-26 mmHg. U.S. Pat. No. 4,857,636 disclosed an intramolecularly crosslinked hemoglobin wherein the subunits of hemoglobin were linked together with o-raffinose, and the p50 was reported to be in the range of 24-32 mmHg. U.S. Pat. No. 5,438,041 disclosed an emulsified hemoglobin wherein the hemoglobin molecules are emulsified in oil by microfluidization, and the p50 was reported to be around 26 mmHg. U.S. Pat. No. 5,618,919 disclosed a polymerized hemoglobin wherein the hemoglobins are intermolecularly polymerized with glutaraldehyde, and the p50 was reported to be 24-32 mmHg.

In summary, the related prior arts have indicated utility of p50 well outside of the p50 of the present invention.

TABLE 2
Examples of Other Modified Hemoglobins Without
Use of PEG as Modifier.

| U.S. Pat. No. | Modified Hemoglobin | p50 (mmHg) |
|---|---|---|
| 4,831,012 | Hemoglobin intramolecularly crosslinked | 20-26 |
| | with diasprin | |
| 4,857,636 | Hemoglobin intramolecularly crosslinked | 24-32 |
| | with oraffinose | |
| 5,438,041 | Emulsified hemoglobin | 26 |
| 5,618,919 | Hemoglobin polymerized with | 24-32 |
| | glutaraldehyde | |

The present inventors have endeavored further to develop an efficient method for the delivery of oxygen to cells and tissues of hypoxia or ischemia and established the useful relationship between the relatively low p50 and the characteristic of selective oxygen delivery to ischemia for the first time in the present invention.

The present invention, however, utilizes mPEG-succinimidyl propionate (SP-PEG) for modification of hemoglobin. The present invention is also unique in that a specific range of p50 is identified where the characteristic oxygen delivery behavior of the present invention is best realized. It was in this relatively narrow p50 range of 6-14 mmHg where the selective oxygen delivery to ischemia was properly demonstrated. We have found that an inventive PEG conjugated-hemoglobin solution for treatment of stroke.

### SUMMARY OF THE INVENTION

Accordingly, it is an object of the present invention a method for treating symptoms such as stroke, comprising an intravascular administration of therapeutically appropriate amount of polyethylene glycol-hemoglobin solution to the mammal for delivering oxygen to cells and tissues of hypoxia or ischemia.

### BRIEF DESCRIPTION OF THE DRAWINGS

The above and other objects and features of the present invention will become apparent from the following description of the invention taken in conjunction with the following accompanying drawings; which respectively show:

FIG. 1: ¹H-NMR data of PEG-conjugated bovine hemoglobin solution;

FIG. 2: RP-HPLC data of PEG-conjugated hemoglobin solution;

FIG. 3: oxygen dissociation curve and p50 value;

FIG. 4: a comparison of latency to find the hidden platform in the Morris water maze study using cerebral thromboembolism rat models;

FIG. 5: a comparison of total distance to find the hidden platform in the Morris water maze study using cerebral thromboembolism rat models;

FIG. 6: a comparison of rearing and leaning counts in the open field study using cerebral thromboembolism rat models;

FIG. 7: a comparison of total distance in the open field study using cerebral thromboembolism rat models;

FIG. 8: a comparison of total infarct volume corrected for brain edema of cerebral thromboembolism rat models;

FIG. 9: a comparison of tissue edema (% edema) expressed as the percent increase in size of the ipsilateral (occluded) hemisphere over the contralateral (untreated) hemisphere of cerebral thromboembolism rat models;

FIG. 10: a representative TTC (2,3,5-triphenyltetrazolium chloride) staining of coronal brain from non-operation group (negative control);

FIG. 11: a representative TTC staining of coronal brain from cerebral thromboembolism rat models injected with saline after injection of blood clot;

FIG. 12: a representative TTC staining of coronal brain from cerebral thromboembolism rat models injected with PEG-hemoglobin SB1 solution, 240 mg/kg after injection of blood clot;

FIG. 13: a representative TTC staining of coronal brain from cerebral thromboembolism rat models injected with PEG-hemoglobin SB1 solution, 480 mg/kg after injection of blood clot; and

FIG. 14: a representative TTC staining of coronal brain from sham-operation group.

### DETAILED DESCRIPTION OF THE INVENTION

The present invention provides the method for treating the symptoms such as stroke, comprising intravenously administering polyethylene glycol-modified hemoglobin solution to the mammal for delivering oxygen to the cells and tissues of hypoxia and ischemia.

In some embodiments, the polyethylene glycol-modified solution comprises polyethylene glycol derivatives such as methoxypolyethylene glycol-succinimidyl propionate, preferably its molecular weight is between 1,000 and 100,000 for modification of hemoglobin and hemoglobin obtained from human, animal such as bovine, porcine and the like, transgenic or recombinant sources.

The above polyethylene glycol-modified hemoglobin solution has an average concentration of between 7 and 13 g/dl, an average molecular weight of from approximately 100 to 140 kDa, a viscosity of approximately 7 cp and an acidity of pH 7.0-8.0.

In some embodiments, the inventive polyethylene glycol-modified hemoglobin solution comprises oxygen affinity higher than that of blood. Above solution of the present invention has a p50 of between 6 and 14 mmHg.

The term ‘p50’ is defined as the partial pressure of oxygen (mmHg) at which point 50% of the hemoglobin is oxygenated. The p50 of normal red blood cell is 26-28 mmHg. Lower p50 such as 6-14 mmHg represents a stronger affinity of hemoglobin for oxygen, thereby the oxygenated hemoglobin is more reluctant to release oxygen.

Above solution has a final hemoglobin concentration of between 4 to 6 g/dl.

Moreover, a degree of above polyethylene glycol conjugated to hemoglobin is between 5 and 15.

In some embodiment, the therapeutically appropriate amount of polyethylene glycol-modified solution is between 96 and 1,920 mg/kg (1 and 20 ml/kg) and it is administered via intravenous injection, thereby deliver oxygen to cells and tissues. In other embodiments, the mammal is a human, preferably who has a symptom such as stroke, hypoxia, ischemia, anemia and the like.

In the efficacy studies for the present invention using animal stroke simulation models, the PEG-hemoglobin of the present invention demonstrated its capability of selective oxygen delivery to ischemia in the study where brain sections from the studied animals were stained to identify either the presence or absence of cellular hypoxia. Also for overall physiology, the PEG-hemoglobin of the present invention demonstrated its capability to restore the studied animals' physical ability as investigated in the water maze tests and the open field tests.

The following Examples are intended to further illustrate the present invention without limiting its scope. The PEG-hemoglobin solution used in the present invention was manufactured by SunBio, Inc. located in South Korea.

### EXAMPLE 1

### Preparation of Native Hemoglobin

Whole Blood Collection and Blood Safety Tests

The hemoglobin can be obtained from human, animal, or recombinant sources. Human hemoglobin can be collected from human whole blood which has been freshly drawn or obtained from out-dated blood from blood banks. Animal-originated hemoglobins may include bovine or porcine sources. The recombinant hemoglobins can be obtained from transgenic herds or cells, or by protein engineering. After collection of whole blood, blood safety test is performed. As for the bovine blood, viral disease causing agents derived from animals include bovine viral diarrhea disease virus(BVDV), parainfluenza virus type 3(PIV-3), infectious bovine rhinotracheitis virus(IBRV), bovine ephemeral fever virus(BEFV), akabane disease virus(AKAV), and ibaraki disease virus(IBAV) are to be screened. Freshly drawn whole blood was examined for virus contamination in comparison to positive control groups comprising 100 and 1,000 TCID₅₀(50% Tissue Culture Infectious Dose) titer of BVDV, PIV-3, IBRV, BEFV, AKAV, and IBAV. In the course of testing, MDBK(Madin-Darby bovine kidney) cell line was used as host cell line for BVDV, IBRV, and PIV-3 and Vero cell line for BEFV, AKAV, and IBAV. Those cells infected by 6 different strains of positive control viruses, respectively, and the blood samples were monitored for developing CPE(cytopathic effect). If CPE develops within 14 days, those groups were determined to be positive. For the negative groups, this study was repeated and then the evaluation was confirmed. Nucleic acid amplification testing, known by its abbreviation NAT to detect viruses and other pathogens in blood, was performed only when the CPE assay of BVDV is ambiguous via RT-PCR (Reverse Transcriptase-Polymerase Chain Reaction). When it comes to bovine blood, BSE(Bovine Spongiform Encephalopathy) safety is a critical factor. BSE is mainly characterized by absence of conventional infective agents (viruses, bacteria, and any known pathogens) and progressive accumulation in the central nervous system of an abnormal form of the Prion protein(PrP) called PrPres that have increased resistance to proteinase K. The Platelia® BSE detection kit (BioRad, U.S.A.) allowed quantitative determination of PrPres on samples of brainstem taken from cattle. Brainstem tissue from the cattle, from which whole blood is to be collected, was subjected to the BSE safety. The test was repeated 3 times. Brainstem samples with an optical density less than the cut-off value, which was calculated from the mean optical density of the four negative control groups, were considered to be negative (it means free of BSE). And whole blood was also confirmed to be free of disease-causing Brucella abortus and Mycobacterium bovis.

Red Blood Cell(RBC) Separation and Washing

Whole blood delivered to Class 100 facility was aliquoted in 500 ml polypropylene tube and then fractionated via centrifugation at 4° C., 5000 rpm for 23 minutes. The supernatant (serum) and buffy coat (white blood cells) were discarded and the remaining red blood cells (RBCs) was transferred to a 20 L glass bottle. Then, equal volume of isotonic saline buffer (NaCl 150 mM, KH₂PO₄ 2 mM and Na₂HPO₄ 8 mM, pH 7.6) was added into the bottle and mixed. And 5 micron hepa filtration (Millipore, U.S.A.) was followed to remove blood clots and other cell debris. In the process of hepa filtration, a filter housing equipped with a 5 micron filter cartridge was sterilized and then washed by consuming 20 L of PFW (pyrogen-free water). After washing, five liters of isotonic saline buffer was used to fill the filter housing and therefore to prevent the lysis of hemoglobin during the filtration. For the purpose of RBC washing, the filtrate was then filtered through 0.1 micron membrane filter (SK Chemicals, Korea). The filter was pretreated with 0.1N NaOH in recirculating manner for 15 minutes and then with 20 L of PFW. Both steps were repeated and this time a 40 L of PFW was consumed for washing. After washing, pH value of the eluant was near 7.6. Then, the RBC solution was connected to the 0.1 micron filter that RBC was forced to recirculate without filtering through the membrane, while the impurities were removed as filtrate. The RBC washing step continued until 40 L of isotonic saline buffer was consumed. The final volume of RBC solution was 10 L.

Hemoglobin Extraction from RBC and Concentration

The 0.1 micron membrane filter (SK Chemicals, Korea) was also used for extraction of hemoglobin in the same manner as in the RBC washing step. A 2.5 L of PFW was added to the 10 L of RBC solution. The filter was pretreated with 0.1N NaOH in circulating manner for 15 minutes and 20 L of PFW. Both steps were repeated and this time a 40 L of PFW was consumed for washing. After washing, pH value of the eluant was near 7.6. Then, the RBC solution was connected to the 0.1 micron filter so that RBC was recirculated. To extract the hemoglobin from RBCs, 60 L of hypotonic buffer I (NaCl 65 mM, KH₂PO₄ 2 mM and Na₂HPO₄ 3 mM, pH 8.2) was added to the RBC reservoir, which made the RBC cell membrane porous. Filtration continued until the total volume in the RBC reservoir was exhausted up to 10 L, same as initial volume. In order to concentrate the hemoglobin extracted from RBC, MWCO (Molecular Weight Cut-Off) 50,000 KrossFlo ultrafiltration membrane (UF membrane, Cat. No. K25S-300-01N, Spectrum, U.S.A.) was used. The preparation procedure for MWCO 50,000 ultrafiltration membrane was identical to that for the 0.1 micron filter. Then, the reservoir containing extracted hemoglobin was connected to the MWCO 50,000 UF membrane so that native hemoglobin was recirculated and concentrated to 5.5±0.3 g/dl. The native hemoglobin was then stored at −20° C. until needed for next step of preparation.

### EXAMPLE 2

### Preparation of PEG-hemoglobin SB1

Chromatographic Purification of the Extracted Hemoglobin

Two anion exchange columns, Matrex PEI-1000 (crosslinked polyethyleneimine, mean particle size: 50 micron, pore size: 1000 Å, Millipore, USA) and Matrex Cellufine Q-500(crosslinked cellulose beads, particle size: 53-125 micron, exclusion limit: >500 kD, Millipore, USA), comprised chromatographic purification of the hemoglobin. Column packing procedure for PEI-1000 was as follows. To make 2 L of packed resin volume, 800 g of PEI resin media was swelled in 4 L of PFW for 1 hour and then, supernatant was discarded. This swelling step was repeated 3 times and in the final step resin was degassed under vacuum. PEI resin slurry was poured into the column. And with the inlet open to release air, top adjuster was inserted and assembled at the slurry interface. For the purpose of equilibration and depyrogenation, the column was flushed with 3 bed volumes of 1.5M NaCl and 0.5N NaOH at a flow rate of 2 bed volumes/hr and then followed by 50 bed volumes of hypotonic buffer II (NaCl 65 mM, KH₂PO₄ 2 mM and Na₂HPO₄ 3 mM, pH 7.6) at a flow rate of 1 bed volume/hr. And this equilibration step continued till the eluent of the column reached to pH 7.6±0.2. The endotoxin content of eluent should be less than 0.03 EU/ml to initiate the purification. In case of Q-500 column packing procedure was as follows. A 2 L of Q-500 resin slurry was carefully poured into the column. And the column was washed with 2 bed volumes of PFW to make 2 L of packed resin volume. For the purpose of equilibration and depyrogenation, the column was flushed with 3 bed volumes of 1.2M NaCl and 0.5N NaOH at a flow rate of 2 bed volumes/hr, respectively and then followed by 30 bed volumes of hypotonic buffer II (NaCl 65 mM, KH₂PO₄ 2 mM and Na₂HPO₄ 3 mM, pH 7.6) at a flow rate of 1 bed volume/hr. And this equilibration step continued till the eluant of the column reached to pH 7.6±0.2. The endotoxin content of eluent should also be less than 0.03 EU/ml to initiate the purification. Following the column preparation, 3 bed volumes of the native hemoglobin (5.5 g/dl, 6 L) were loaded onto the PEI-1000 column. And the eluent from PEI was connected to be loaded continuously onto the Q-500 column. The flow rate in process of purification was 1 bed volume/hour(33 ml/minute). These two anion exchange chromatography processes of the invention were able to eliminate phospholipids, endotoxins, and residual DNA from the hemoglobin solution.

Virus Removal Step from the Hemoglobin

In process of nanofilter preparation, a filter housing equipped with a DV20 filter cartridge was sterilized and then followed by filter wetting with 2 L of 30% isopropyl alchol and flushed with excess volume of PFW. After flushing, five liters of hypotonic buffer II (NaCl 65 mM, KH₂PO₄ 2 mM and Na₂HPO₄ 3 mM, pH 7.6) was added to fill the filter housing and therefore to prevent the reduction of electrolyte level in the hemoglobin solution. The column flow-through (7 L) collected from the purification step was then passed through the virus removal filter having a pore size of 20nm (DV20, Pall Filtron Corp., USA.) at a flow rate of 150 ml/min. The fluid pressure was controlled to be within 1.5±0.5 bar.

PEG Conjugation to the Hemoglobin

A 7 L of hemoglobin solution (about 4.5 g/dl) was collected after nanofiltration. The pH of hemoglobin solution was adjusted to 8.2-8.4 with the 1N NaOH. And then methoxypolyethylene glycol-succinimidyl propionate (SP-PEG, MW: 5000, SunBio, Korea) was added to the purified hemoglobin solution. Initially a 12 equivalent of SP-PEG was added to the hemoglobin solution, and the pH of the mixture was maintained at 8.2-8.4. The conjugation was carried out at ambient temperature for a few hours. And the degree of PEG conjugation to the hemoglobin was monitored with GPC (Gel Permeation Chromatography). In the preferred embodiment, the molar ratio of SP-PEG to purified hemoglobin was between 18:1 and 24:1. This molar ratio range routinely resulted in 9-11 PEG molecules conjugated to one molecule of hemoglobin. Preferably the molecular weight for SP-PEG was between 2,500 and 40,000 Da. The amount of SP-PEG to be added to the hemoglobin was determined as follows: {Hemoglobin concentration(g/L)/Molecular weight of hemoglobin(Da)}×{Hemoglobin volume(L)}×({Molecular weight of SP-PEG(Da)}×(Molar ratio) ×(1/% of SP-PEG purity).

Ultrafiltration(MWCO 50,000) to Remove Unreacted PEG and Unmodified Hemoglobin

The preparation procedure for MWCO 50,000 ultrafiltration membrane(Cat. No. K25S-300-01N, Spectrum, USA) was as follows. The filter was treated with 0.1N NaOH in recirculating manner for 15 minutes and then with 20 L of PFW. Both steps were repeated and as repeated, 40 L of PFW was consumed for washing. After washing, pH value of the eluent was 7.6. The PEG-conjugated hemoglobin solution was then filtered with a ultrafiltration cartridge, recirculating between the ultrafiltration device and reaction mixture. Concurrently, impurities including the residual PEG and unmodified hemoglobin were discarded as filtrate. Ultrafiltration step continued until 40 L of hypotonic buffer II(NaCl 65 mM, KH₂PO₄ 2 mM and Na₂HPO₄ 3 mM, pH 7.6) was consumed.

Reduction of Methemoglobin

During the manufacturing process of PEG-hemoglobin, methemoglobin level may increase. Methemoglobin carries Fe³⁺ ion and thus unable to deliver oxygen. In order to maximally facilitate oxygen delivery by PEG-hemoglobin, methemoglobin formation needs to be suppressed. In preferred embodiments of the invention, D,L-cysteine (M. W. 121.2, Sigma, USA) was used as reducing agent for methemoglobin. Ten equivalents of D,L-cysteine to the PEG-hemoglobin was added. Reduction reaction was allowed to proceed at pH 7.6±0.1, for between 6 and 12 hours, at 4° C. The amount of D,L-cysteine to be added to the PEG-conjugated hemoglobin solution was calculated as follows: {Hemoglobin concentration(g/L)/Molecular weight of hemoglobin(Da)}×(Molecular weight of D,L-cysteine)×{Hemoglobin volume(L)}×(Molar ratio).

Ultrafiltration (MWCO 50,000) to remove D,L-cysteine The preparation procedure for MWCO 50,000 ultrafiltration membrane(Cat. No. K25S-300-01N, Spectrum, USA) was same as mentioned above. The filter was treated with 0.1N NaOH in recirculating manner for 15 minutes and then with 20 L of PFW. Both steps were repeated and as repeated, and a 40 L of PFW was consumed for washing. The reaction mixture including D,L-cysteine was then recirculated through a ultrafiltration cartridge from which the cysteine was filtered out. The ultrafiltration step continued until 60 L of hypotonic buffer II (NaCl 65 mM, KH₂PO₄ 2 mM and Na₂HPO₄ 3 mM, pH 7.6) was consumed.

Removal of Endotoxin from PEG-conjugated Hemoglobin Solution

In order to additionally remove endotoxin from PEG-hemoglobin solution, Matrex Cellufine Q-500 column was used once more. Column packing procedure for Q-500 was identical to the above-mentioned except the packing volume. One liter of Q-500 resin slurry was carefully poured into the column. And the column was washed with 2 bed volumes of PFW to make 1 L of packed resin volume. For the purpose of equilibration, the column was flushed with 3 bed volumes of 1.2M NaCl and 0.5N NaOH at a flow rate of 2 bed volumes/hr, respectively and then followed by 30 bed volumes of hypotonic buffer II (NaCl 65 mM, KH₂PO₄ 2 mM and Na₂HPO₄ 3 mM, pH 7.6) at a flow rate of 1 bed volume/hr. And this equilibration step continued till the eluent of the column reached to pH 7.6±0.2. The endotoxin content of eluent should also be less than 0.03 EU/ml to initiate the process of endotoxin removal. Following the column preparation, 3 bed volumes of PEG-hemoglobin solution were loaded onto the Q-500 column. The flow rate in process of endotoxin removal was 1.5 bed volume/hour.

Filter Sterilization of PEG-Conjugated Hemoglobin Solution

The electrolytes formulating the PEG-hemoglobin solution were to be 115±15 mM of Na⁺, 3±2 mM of K⁺, and 105±15 mM of Cl⁻. And to fulfill the specification, a total appropriate amount of 45 mM NaCl was added to the PEG-hemoglobin solution obtained from the endotoxin removal step. And the solution was then passed through the 0.22 micron membrane filter (Millipak 200, Millipore, USA) for filter sterilization. The final product was aliqouted in a PVC blood bag (Becton Dickinson, USA) to a volume of 150 ml each bag and stored at −20° C.

### EXAMPLE 3

### Characterization of PEG-Hemoglobin SB1

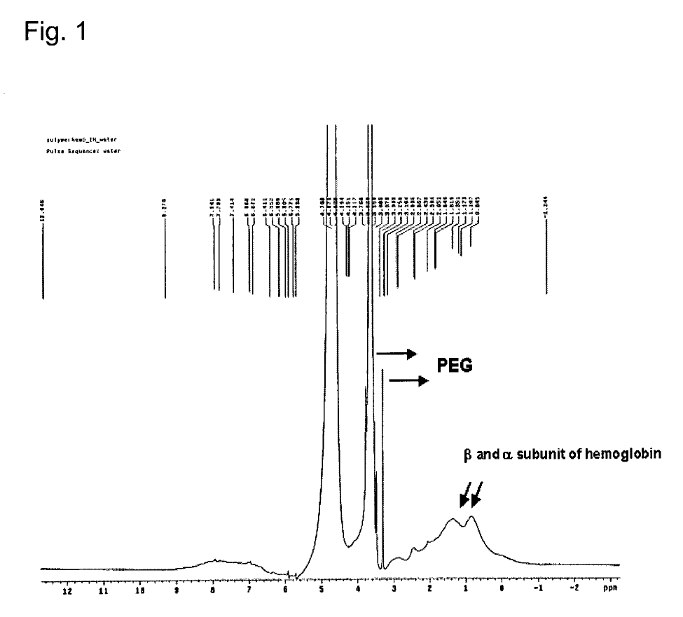

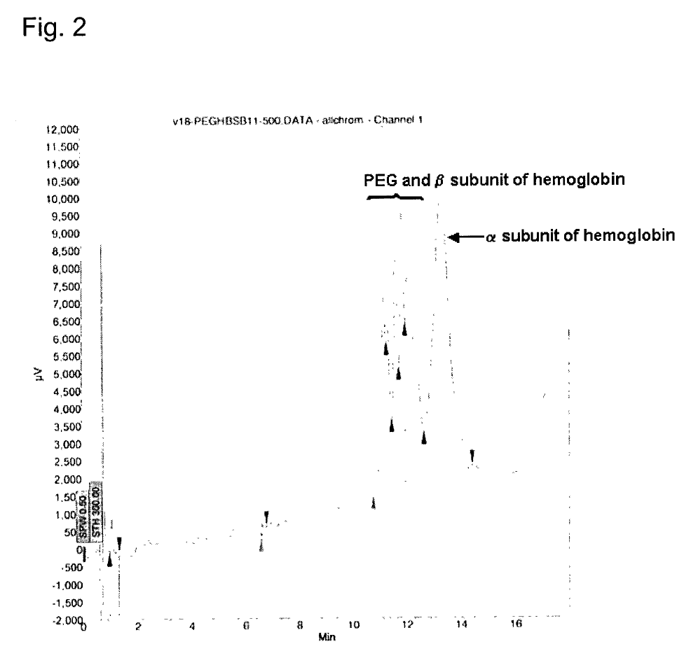

PEG-conjugated hemoglobin SB1s were confirmed by ¹H-NMR(Nuclear Magnetic Resonance) spectroscopy (FIG. 1). And inventive solution was conducted to RP-HPLC (Reverse Phase-High Performance Liquid Chromatography, column; Alltech Prosphere C18 (250×4.6 mm), mobile phase; 0.01% trifluoroacetic acid in actonitrile and 0.01% trifluoroacetic acid in distilled water with a gradient)(FIG. 2). The purpose of proton NMR analysis was to confirm the overall structure of hemoglobin whether it was changed or not even after PEG modification. The figure represented the hemoglobin kept its tertiary structure intact as detected by β and α subunit from 2-0 ppm, when compared to native hemoglobin. In the RP-HPLC analysis, structural change resulting from polarity difference was monitored. The fact that the peak pattern where β and α subunit was detected sequentially and retention time of the PEG-hemoglobin SB1 was consistent with the native hemoglobin represented PEG modification did not induce tertiary structure disturbance.

Molecular weights of PEG-hemoglobin SB1s were determined by SDS-PAGE (Sodium Dodesyl Sulphate-Polyacrylamide Gel Electrophoresis) techniques under reducing and non-reducing conditions and also could be determined by MALDI-TOF technology (Matrix-assisted laser/desorption ionization time of flight analyzer). The hemoglobin concentration was analyzed by 912 CO-Oxylite machine (AVL Scientific Corporation, U.S.A.). The degree of PEG modification was calculated by TNBS assay (2,4,6-trinitrobenzenesulfonic acid, Anal. Biochem., 14, pp 328-336, 1966). And the PEG-hemoglobin concentration was determined by the following calculation:

(mean No. of PEG per Hb×5,000+64,500)/64,500×Hb concentration(g/dl).

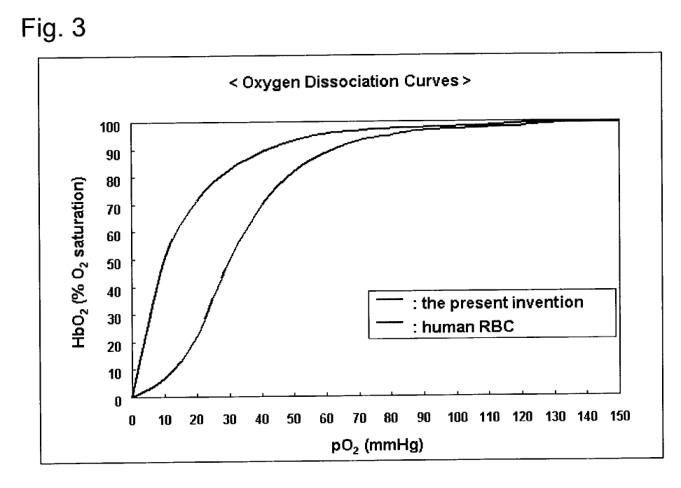

The oxygen dissociation curve of the present invention and human RBC is plotted in FIG. 3. The oxygen dissociation curve was measured and plotted by using a Hemox-Analyzer. The curve represents the pattern and behavior of oxygen carriers such as human red blood cell (RBC) and the present invention as plotted HbO₂ (oxygenation degree of hemoglobin) versus pO₂ (partial pressure of oxygen in the surrounding). The fact that the oxygen dissociation curve of the present invention is left-shifted compared to human RBC demonstrates the stronger oxygen affinity of the present invention, thereby making possible the characteristics of the present invention.

The composition of electrolyte was analyzed by 9180 Electrolyte Analyzer (AVL Scientific Corporation). Endotoxin levels were measured by the Limulus Amebocytic Lysate (LAL) assay. The endotoxin levels were established with reference to measurements made with gel clot assays or kinetic turbidometric assays. The results of above examinations are shown in Table 3.

TABLE 3
Characteristics of PEG-hemoglobin SB1

| Molecular weight | | 110 ± 10 kDa |
|---|---|---|
| Hemoglobin concentration | | 5.1 ± 0.7 g/dl |
| Degree of PEG modification | | 9 ± 2 |
| PEG-hemoglobin concentration | | 9 ± 1.2 g/dl |
| p50 | | 10 ± 2 mmHg |
| Viscocity | | 7 cp |
| Half-life | | 24 hrs (rat), 48 hrs (dog) |
| Electrolyte | Na⁺ | 115 ± 15 mM |
| | K⁺ | 3 ± 2 mM |
| | Cl⁻ | 105 + 15 mM |
| PO³⁻₄ | | 1-5 mM |
| Color | | Deep red |
| pH | | 7.5 ± 0.2 |
| Endotoxin | | Less than 0.25 EU/ml |

### Example 4

### Establishment of Rat Cerebral Thromboembolism Model

I. Animals

Sprague-Dawley(SD) rats weighing 350-400 g(n=50) were used. The rats were housed in a 12 hours light:dark cycle and had free access to food and filtered water.

II. Preparation of Blood Clot

Thrombin (12 U/0.2 ml saline) was retained in a 1 ml syringe and a 0.8 ml of blood from a SD rat was withdrawn into the other 1 ml syringe. The two syringes were interconnected by a PE (polyethylene)-10 tube (inner diameter 0.28 mm). The clot was formed and shifted by continuous alternating movement from one syringe to the other for 3 minutes and stored for 30 minutes in situ. III. Rat cerebral thromboembolism model The animal was anesthetized with 1.5% enflurane and maintained under 30:70 oxygen/nitrous oxide with a face mask. The right common carotid arteries (CCA), the right external carotid artery (ECA) and the internal carotid artery (ICA) were isolated via a midline incision. A 4-0 silk suture was loosely tied at the origin of the ECA and ligated at the distal end of the ECA. The right CCA and ICA were temporarily clamped using a bulldog clip. A PE catheter filled with a 200μl clot was introduced into the ECA lumen and then was gently advanced from the ECA into the lumen of the ICA. Test material (PEG-hemoglobin in the present invention) was infused, through the catheter initially equipped in the course of surgery, into the right femoral vein 5 minutes after the clot injection. Infusion was performed using infusion pump at 5 ml/kg/10 min.

TABLE 4
Rat Thromboembolism Model Study Test Group Assignment

| | | Infusion | Infusion | No. of | |
|---|---|---|---|---|---|
| | | dose | volume | Subjects | Subjects |
| Groups(G1-G5) | | (mg/kg) | (ml/kg) | (rats) | Labeling |
| G1 | Negative control group | 0 | 5 | 5* (5⁺) | M01-M05 |
| | (Saline: non-operation) | | | | (M06-M10) |
| G2 | Positive control group | 0 | 5 | 5* (5⁺) | M11-M15 |
| | (Saline: | | | | (M16-M20) |
| | thromboembolism) | | | | |
| G3 | Test group | 240 | 2.5 | 5* (5⁺) | M21-M25 |
| | (PEG-hemoglobin, low | | | | (M26-M30) |
| | dose) | | | | |
| G4 | Test group | 480 | 5 | 5* (5⁺) | M31-M35 |
| | (PEG-hemoglobin, high | | | | (M36-M40) |
| | dose) | | | | |
| G5 | Sham operation | 0 | 0 | 5* (5⁺) | M41-M45 |
| | | | | | (M46-M50) |

*TTC staining group,
⁺Morris water maze and open field method groups

### EXAMPLE 5

### Morris Water Maze Study

I. Materials and methods

This study used a round pool (diameter 120 cm, height 30 cm) of water in which a platform (diameter 10.6 cm) was placed. During the study, an animal was placed in 10 one quadrant of the pool and allowed to swim until the platform was found. Within 90 seconds of time set point, latency and distance for the animals to reach platform were tracked by a video camera and analyzed by video track software. Morris water maze study was performed for animals rested for 3 days post-surgery, and the study was performed 4 times a day for 4 days and the data were collected and evaluated. 15

II. Results

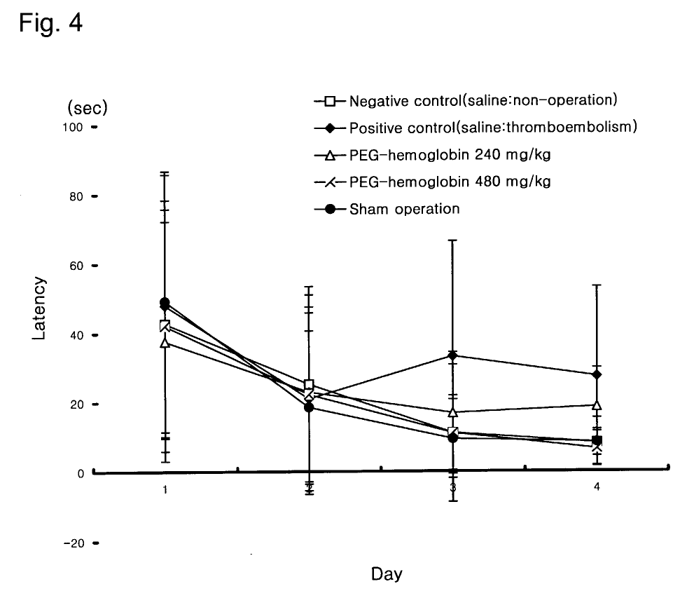

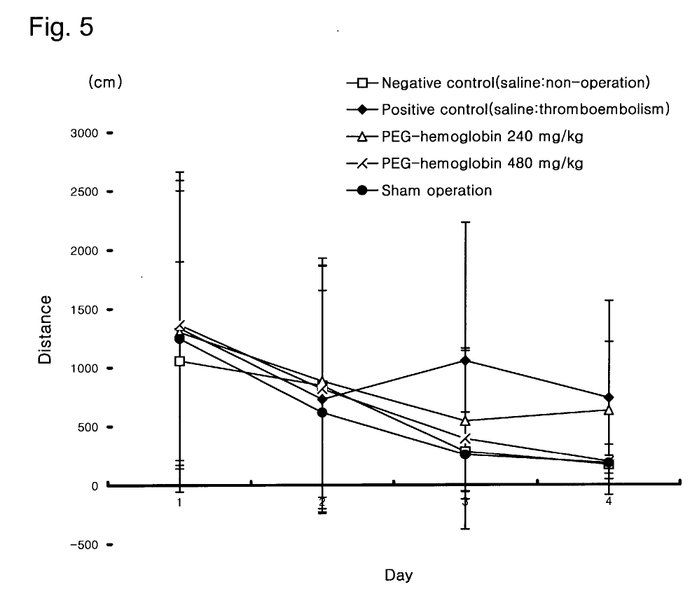

In the negative control group (saline, non-operation), the time required to find the platform was shortened from 42.75±33.05 seconds on day 1 to 8.31±3.91 seconds on day 4 and also the distance was decreased from 1056.17±845.49 cm to 169.39±75.58 cm. And, similarly, although in positive control group which had thromboembolism and then saline buffer was infused, the time required to find the platform was shortened from 47.97±37.83 sec to 27.52±25.79 sec and also the distance was decreased from 1336.44±1164.78 cm to 738.29±825.18 cm, but the reduction level was significantly low and when compared with the negative control group, time and distance were rather increased from day 2 to day 3 and 4. In the sham operation group, similar to negative control group, the time to reach the platform and total distance were reduced gradually. Almost comparable data were also collected from the test groups where PEG-hemoglobin was infused. In the high dose PEG-hemoglobin test group (480 mg/kg), the time and distance were even more decreased than those of negative control group. However, in the low dose PEG-hemoglobin test group (240 mg/kg), no significant difference between data collected from day 3 and from day 4 was noted. Compared with the positive control group, until the day 2, the difference among all groups was minimal. But on day 3 and day 4 the time and distance were significantly reduced in the high dose PEG-hemoglobin test group and sham operation group. Numeric data on the latency and total distance to find the hidden platform in the Morris water maze study of cerebral thromboembolism rat models are summarized in Table 5 and shown in FIG. 4 and FIG. 5.

TABLE 5
Morris Water Maze Test Results

| Behavioral | | | | | | | | | |
|---|---|---|---|---|---|---|---|---|---|
| variable | Day | G1(n = 5) | G2(n = 5) | | G3(n = 5) | G4(n = 5) | | G5(n = 5) | |
| Latency | 1 | 42.75 ± | 47.97 ± | | 37.60 ± | 42.13 ± | | 49.17 ± | |
| (seconds) | | 33.05 | 37.83 | | 34.58 | 36.23 | | 37.60 | |
| | 2 | 25.24 ± | 21.05 ± | | 22.98 ± | 22.25 ± | | 18.55 ± | |
| | | 28.18 | 26.52 | | 22.87 | 28.77 | | 22.14 | |
| | 3 | 11.16 ± | 33.28 ± | # | 16.90 ± | 11.09 ± | *** | 9.42 ± | *** |
| | | 10.78 | 33.18 | | 17.60 | 19.83 | | 11.39 | |
| | 4 | 8.31 ± | 27.52 ± | ## | 18.68 ± | 6.60 ± | *** | 8.51 ± | *** |
| | | 3.91 | 25.79 | | 11.30 | 5.03 | | 7.01 | |
| Total | 1 | 1056.17 ± | 1336.44 ± | | 1301.91 ± | 1364.44 ± | | 1401.84 ± | |
| distance | | 845.49 | 1164.78 | | 1359.52 | 1224.76 | | 1143.30 | |
| (cm) | 2 | 843.02 ± | 727.19 ± | | 882.78 ± | 815.94 ± | | 614.95 ± | |
| | | 1084.77 | 927.46 | | 986.23 | 1046.63 | | 703.90 | |
| | 3 | 282.64 ± | 1054.55 ± | # | 542.01 ± | 391.29 ± | ** | 256.43 ± | ** |
| | | 334.05 | 1175.31 | | 600.62 | 769.59 | | 278.70 | |
| | 4 | 169.39 ± | 738.29 ± | ## | 632.22 ± | 194.30 ± | *** | 183.26 ± | *** |
| | | 75.58 | 825.18 | | 582.74 | 147.81 | | 124.73 | |

Results are mean ± S.D.
#: Significantly different from the negative control, p < 0.05.
##: Significantly different from the negative control, p < 0.01.
**: Significantly different from the positive control, p < 0.01.
***: Significantly different from the positive control, p < 0.001.
G1: non-operation,
G2: saline(thromboembolism),
G3: PEG-hemoglobin 240 mg/kg
G4: PEG-hemoglobin 480 mg/kg,
G5: sham operation group

III. Discussion

In the Morris water maze study, as normal rats have learning and memory abilities, it is natural that the time and total distance for the rats to find the platform is reduced as tests are repeated. And this result was consistent with that of the negative control (non-operation, saline infusion) group of this Example. Whereas, the positive control group (thromboembolism surgery and saline infusion) showed similar results only till day 2. From day 3, the time to find the platform was not shortened and furthermore normal behavior like crossing the round pool was rarely noted. This represents that thromboembolism in the positive control group did induce ischemia in the hemisphere of the rat brain and negatively affected the cognitive function including learning and memory. Notably, the PEG-hemoglobin test group demonstrated comparable results as seen in the negative control group. It means that the PEG-hemoglobin of the present invention may be able to restore normal brain functions even after the brain ischemia has occurred. And predictably, in the sham operation group, similarly to the negative control group, the time to reach the platform and total distance were reduced gradually.

### EXAMPLE 6

### Open Field Study

I. Materials and methods

The animals were exposed to open field surrounded by walls, and for 10 minutes, total number of rearing and leaning and total distance traveled in the open field were measured. Rearing and leaning were counted as the number of behavior times which the animal stood on its hind legs with the forelegs in the air or against the wall, respectively. Exposed to novel environment, rats normally get nervous and highly active in motion and show exploratory behavior.

II. Results

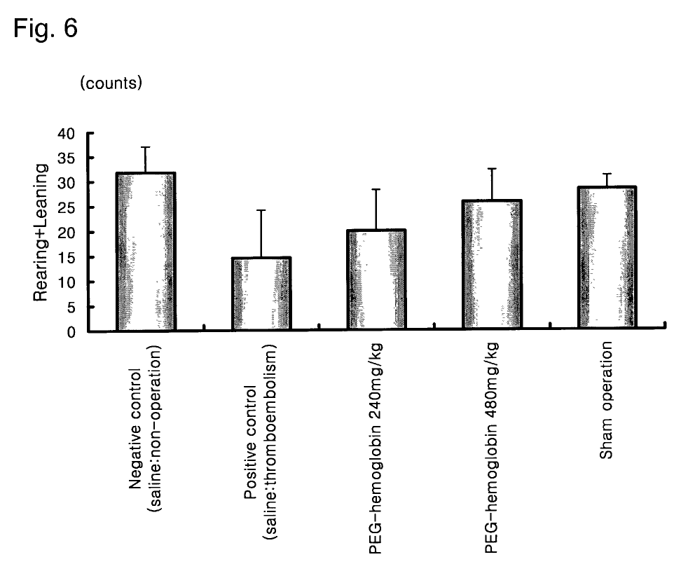

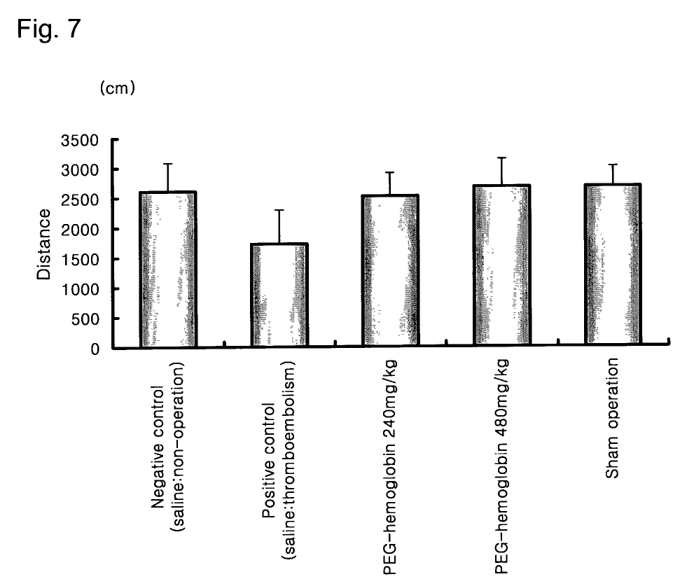

Total number of times combined rearing with leaning was 20.00±8.15 for low dose (240 mg/kg) PEG-hemoglobin group, 25.80±6.42 for high dose (480 mg/kg) PEG-hemoglobin group, and 28.40±2.70 for sham operation group. And the total distance traveled in the open field was 2522.47±385.72 cm for low dose PEG-hemoglobin group, 2678.44±463.84 cm for high dose PEG-hemoglobin group, and 2682.69±334.72 cm for sham operation group. Results from the low dose PEG-hemoglobin, the high dose PEG-hemoglobin, and sham operation group were not significantly different from those of negative control group having values of 31.80±5.26 and 2609.31±473.95 cm, respectively. But compared with the positive control group, low dose PEG-hemoglobin, high dose PEG-hemoglobin, and sham group showed significantly increased values. Results of the locomotor activity in the open field study of cerebral thromboembolism rat models are summarized in Table 6 and shown in FIG. 6 and FIG. 7.

TABLE 6
Open Field Test Results of Thromboembolism Rats

| Behavioral | Group | | | | | | | | |
|---|---|---|---|---|---|---|---|---|---|
| variable | G1(n = 5) | G2(n = 5) | | G3(n = 5) | | G4(n = 5) | | G5(n = 5) | |
| Rearing | 12.60 ± | 5.00 ± | | 3.80 ± | | 7.00 ± | | 6.40 ± | |
| (count) | 7.92 | 8.54 | | 2.59 | | 4.90 | | 3.85 | |
| Leaning | 19.20 ± | 9.60 ± | | 16.20 ± | | 18.80 ± | | 22.00 ± | |
| (count) | 4.09 | 2.97 | | 8.96 | | 4.02 | | 4.36 | |
| Rearing + | 31.80 ± | 14.60 ± | ## | 20.00 ± | | 25.80 ± | | 28.40 ± | * |
| Leaning | 5.26 | 9.56 | | 8.15 | | 6.42 | | 2.70 | |
| Total | 2609.31 ± | 1730.44 ± | ## | 2522.47 ± | * | 2678.44 ± | * | 2682.69 ± | * |
| distance (cm) | 473.95 | 564.92 | | 385.72 | | 463.84 | | 334.72 | |

Results are mean ± S.D.
##: Significantly different from the negative control, p < 0.01.
*: Significantly different from the positive control, p < 0.05.
G1: non-operation,
G2: saline (thromboembolism),
G3: PEG-hemoglobin 240 mg/kg
G4: PEG-hemoglobin 480 mg/kg,
G5: sham operation group

III. Discussion

In the study of open field method, exposed to novel environment, rats normally feel anxiety and then get highly active in motion and show exploratory behavior. Based on these behavioral characteristics, it is possible to evaluate the efficacy of PEG-hemoglobin of the present invention. In regard to the critical behavioral measures, rearing, leaning and total distance traveled in the open field was noted. In the negative control group, total number of times combined rearing with leaning was 31.80±5.26 and total distance was 2609.31±473.95 cm. And the positive control group, compared to negative control group, has reduced values as to 46% for total number of times combined rearing and leaning and to 66% for total distance. This represents that resulting from brain ischemia, rats belonging to positive control group became ignorant and non-adjusting to novel environment. And the low dose (240 mg/kg) PEG-hemoglobin group, the high dose (480 mg/kg) PEG-hemoglobin group and sham operation group, compared to negative control group, showed slightly reduced or almost comparable values as to 63%, 81%, and 89% for total number of times combined rearing and leaning, respectively, and as to 97%, 103%, and 103% for total distance, respectively. This indicates that PEG-hemoglobin groups and sham operation group regained normal behavior and locomotor activity.

### EXAMPLE 7

### Measurement of Infarction Induced by the Thromboembolism Model

I. Materials and Methods

TTC Staining: In normal tissue, mitochondrial dehydrogenases reduce TTC (2,3,5-triphenyltetrazolium chloride) and then tissue is stained red. On the other hand, ischemic tissue induced by thromboembolism is lost their TTC reducing ability and infarct region is then stained white. To identify and measure ischemic brain damage primarily in rat cerebral thromboembolism model, TTC staining (Bederson et al., Stroke, 17(6), pp 1304-1308, 1986) was used. Two hours after infusion of saline or PEG-hemoglobin SB1, the brain was removed and then incubated in saline buffer at 4° C. for 10 minutes. The brain was then sliced coronally from the frontal pole at a thickness of 2 mm per section with a brain matrix. Consecutively sectioned, seven brain coronal slices were then incubated in 2% TTC solution for 1 hour at 37° C.

Infarct size and edema(%): TTC-stained brain slices were fixed with 10% phosphate-buffered formalin solution. Posterior surface of each stained section was scanned with a flatbed scanner and their images were analyzed to classify the infarct ‘white’ area in the cerebral cortex and striatum versus the normal ‘red’ area. The images were analyzed using an image-processing software, Photoshop 5.0 (Adobe System). To complement the effect of edema incurred in ischemic cerebral hemisphere, corrected infarct volume was calculated.

Total infarct volume (mm³)=sum of infarct area×slice thickness

Corrected infarct region=gross area of contralateral (left) cerebral hemisphere—normal tissue area of ipsilateral (right) cerebral hemisphere

Corrected infarct volume=corrected infarct region×slice thickness

Edema (%)=(A−B)/B×100

A: volume of ischemia-induced cerebral hemisphere in each coronal slices(mm³)

B: volume of normal cerebral hemisphere in each coronal slices(mm³)

II. Results

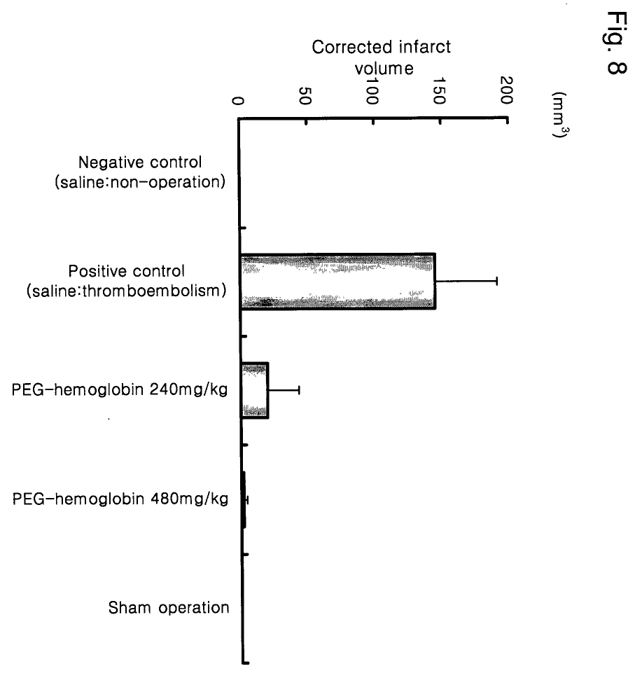

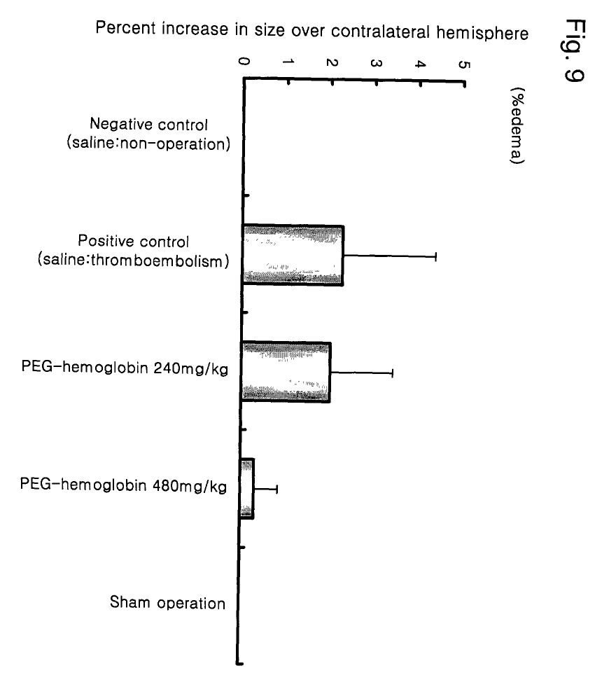

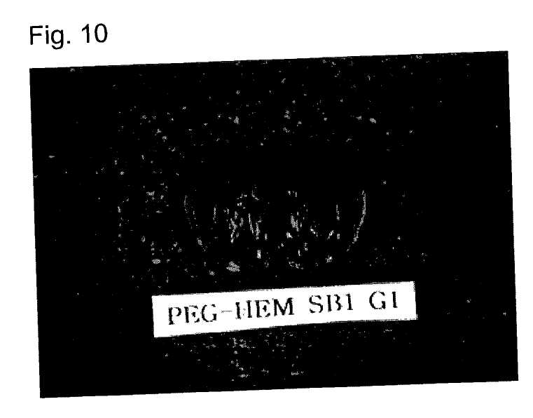

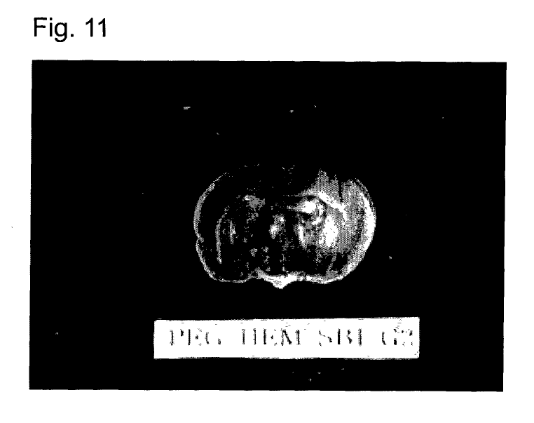

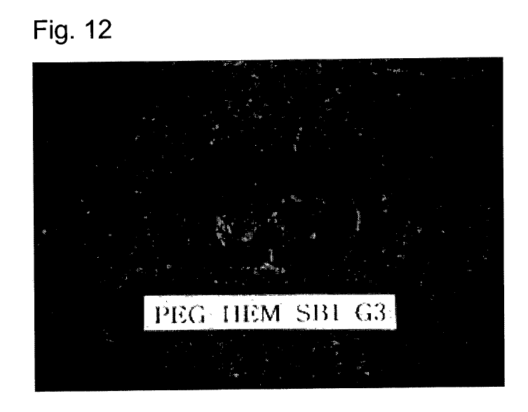

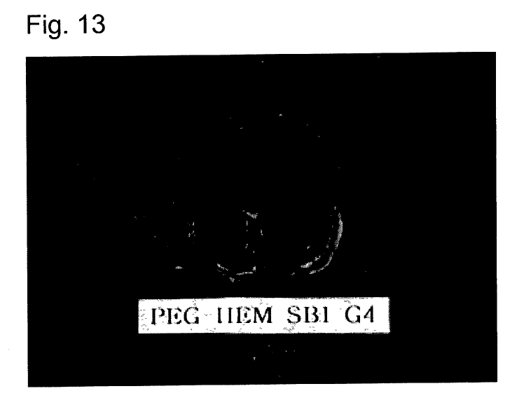

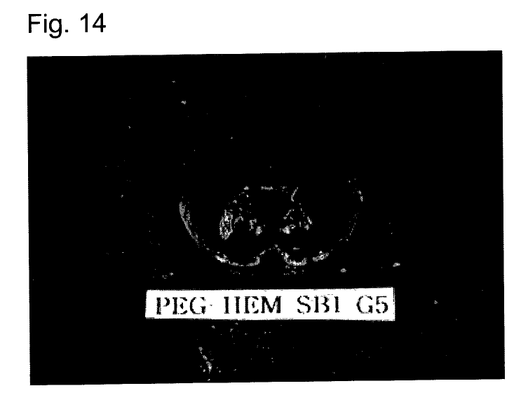

As a result of TTC staining, Infarct was not detected in the negative control group (FIG. 10) and sham operation group (FIG. 14). In the positive control group, the presence of infarct was observed between 7 and 9 mm area from the brain frontal pole (FIG. 11). To compare this to the negative control group, both infarct area and % edema were increased significantly. In the low dose PEG-hemoglobin group (FIG. 12), though infarct was observed in 4 rats from the five rats in total, the infarct area was significantly reduced compared to the positive control group and was almost hard to distinguish infarct from normal region. And in the case of the high dose PEG-hemoglobin group (FIG. 13), although infarct was observed in 2 of the 5 rats in total, the infarct was monitored only in the slice 9 mm away from the frontal pole among 7 slices and the area of infarct was also small. In the infarct volume and corrected infarct volume, low dose, high dose infusion, and sham operation group showed significant reduction in comparison to positive control group. FIG. 8 shows infarct volume corrected for brain edema of cerebral thromboembolism rat model. And in terms of % edema, high dose PEG-hemoglobin group and sham operation group demonstrated significant reduction when compared to the positive control group (FIG. 9).

III. Discussion

By distinguishing normal brain tissue from ischemic tissue via TTC staining and measurement of the area, the efficacy of the PEG-hemoglobin of the present invention in the cerebral thromboembolism model was evaluated. Based on the observation that the infarct volume, the corrected infarct volume, and the % edema of the positive control group were all significantly increased when compared to negative control group, it is confirmed that brain ischemia was successfully induced by the thromboembolism model employed in this invention. Both the low dose PEG-hemoglobin and the high dose PEG-hemoglobin groups, when compared to positive control group, showed significantly reduced infarct volume and corrected infarct volume calculated by complementing the edema resulting from cerebral ischemia. Thus it can be stated that PEG-hemoglobin of the present invention was able to reverse the brain ischemia and restore normal state of the brain.

The invention has been described in particular exemplified embodiments. However, the foregoing description is not intended to limit the invention to the exemplified embodiments, and the skilled artisan should recognize that variations can be made within the scope of the invention as described in the foregoing specification. The invention includes all alternatives, modifications, and equivalents that may be included within the true spirit and scope of the invention as defined by the appended claims.

What is claimed is:

1. A method for treating symptoms by delivering oxygen to cells and tissues comprising administering therapeutically appropriate amount of polyethylene glycol-hemoglobin solution to the mammal, wherein said polyethylene glycol-hemoglobin solution has a concentration of between 7 and 13 g/dl.

2. The method of claim 1, wherein the mammal is a human.

3. The method of claim 1, wherein said symptoms are selected from the group of stroke, hypoxia, ischemia and anemia.

4. The method of claim 1, wherein said amount is between 96 and 1,920 mg/kg (1 and 20 ml/kg).

5. The method of claim 1, wherein said administering is via intravenous injection.

6. The method of claim 1, where said solution has a p50 of between 6 and 14 mmHg.

7. The method of claim 1, wherein a degree of said polyethylene glycol in hemoglobin modification is between 5 and 15.

8. The method of claim 1, wherein said polyethylene glycol is methoxy polyethylene glycol-succinimidyl propionate.

9. The method of claim 8, wherein the molecular weight of said methoxy polyethylene glycol-succinimidyl propionate is between 1,000 and 100,000.

10. The method of claim 1, wherein said hemoglobin is obtained from human.

11. The method of claim 1, wherein said hemoglobin is obtained from animal.

12. The method of claim 1, wherein said hemoglobin is obtained from transgenic.

13. The method of claim 1, wherein said hemoglobin is obtained from recombinant sources.
